(19) United States
(12) Patent Application Publication
(10) Pub. No.: US 2007/0190535 A1
(43) Pub. Date: Aug. 16, 2007

(54) Size fractionation of nucleic acid samples

(76) Inventors: Gerald E. Hall JR., Morrisville, PA (US); Michael T. Barrett, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/354,308

(22) Filed: Feb. 13, 2006

(51) Int. Cl. C12Q 1/68; C07H 21/04

(52) U.S. Cl. 435/6; 536/25.4

(57) ABSTRACT

Methods for size fractionating nucleic acid molecules, using glass fiber filtration columns are described. The methods are useful in many downstream applications including array-based comparative genomic hybridization (aCGH), PCR-based sequencing, somatic genotypic with polymorphic markers, etc.

### BACKGROUND

Genomic DNA copy number variation is a marker for many diseases. For example, the development and progression of human cancer is often accompanied by the accumulation of genetic defects, including chromosomal instability and chromosomal amplification, duplication, deletion or loss. Similarly, developmental abnormalities such as Down's Syndrome, Prader Willi Syndrome, etc. are associated with gain or loss of one copy of a chromosome or chromosomal region. Detecting and mapping copy number are important in studying these disorders and in determining the loci of critical genes that are actively involved in the development and progression of these diseases.

Changes at the genomic level, such as changes in genomic DNA copy number were initially studied by karyotyping, but this process was impeded by the small amount of high-quality metaphase spreads available for analysis. Furthermore, the complex nature of chromosomal changes associated with copy number alterations made traditional methods of karyotyping much more difficult. These methods were ultimately superceded by comparative genomic hybridization (CGH), a method for genome-wide analysis of DNA copy number in a single experiment.

The quality of genomic DNA in a given sample is typically dependent on the efficiency of tissue isolation and subsequent DNA extraction. For example, tissue biopsies obtained under clinical protocols frequently are exposed for varying amounts of time to sub-optimal conditions, such as room temperature, tissue sectioning, etc. These conditions can induce degradation processes such as necrosis or apoptosis. Thus, even under the most stringent protocols, varying amounts of DNA degradation can be introduced into a sample. This is particularly problematic when handling biopsies from enzymatically enriched tissues such as pancreas, stomach, liver, and other regions of the GI tract. These degraded DNA samples are inadequate to serve as templates for amplification by highly processive enzymes.

### SUMMARY

This patent is directed to methods for size fractionation of nucleic acid samples. Embodiments include enriching a nucleic acid sample for desired molecular weight molecules, e.g., high molecular weight fragments, low molecular weight fragments, etc.

The methods described herein use various salt and alcohol concentrations to affect the binding properties of DNA fragments to glass fiber filters. At specific salt and alcohol concentrations, large DNA fragments bind to the glass fibers, while smaller DNA fragments do not bind and pass through the filter in the flow-through fraction.

In another aspect, methods for enriching a sample of genomic DNA with high molecular weight fragments are provided. In an embodiment, the methods described herein are used to collect high molecular weight genomic DNA fragments that are used in different downstream applications, such as aCGH.

Another aspect provides kits that include devices and compositions for separating nucleic acids according to size. The kits include one or more glass fiber filtration devices along with binding buffers and reagents, such as chaotropic salts and lower alcohols needed to separate nucleic acid fragments of different sizes.

### BRIEF DESCRIPTION OF THE DRAWINGS

FIG. 1 shows an electropherogram demonstrating the separation of DNA fragments by size, according to an embodiment described herein.

### DETAILED DESCRIPTION

Various embodiments will be described in detail with reference to the drawings, wherein like reference numerals represent like parts throughout the several views. Reference to various embodiments does not limit the scope of the claims attached hereto. Additionally, any examples set forth in this specification are not intended to be limiting and merely set forth some of the many possible embodiments for the claims.

Unless defined otherwise, all technical and scientific terms used herein have the same meaning as commonly understood to one of ordinary skill in the art. Although any methods, devices and material similar or equivalent to those described herein can be used in practice or testing, the methods, devices and materials are now described.

All publications and patent applications in this specification are indicative of the level of ordinary skill in the art, and are incorporated herein by reference in their entireties.

The term “genome” refers to all nucleic acid sequences (coding and non-coding) and elements present in or originating from a single cell or each cell type in an organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. For example, eukaryotic genomes in their native state have regions of chromosomes protected from nuclease action by higher order DNA folding, protein binding, or subnuclear localization.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of X chromosomes (female) for a total of 46 chromosomes. A genome of a cancer cell may contain variable numbers of each chromosome in addition to deletions, rearrangements and amplification of any subchromosomal region or DNA sequence.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein means a polymer composed of either DNA or RNA, and used as probes to find a complementary sequence of DNA or RNA.

The term “polymerase” refers to an enzyme that links individual nucleotides together into a long strand, using another strand as a template. There are two general types of polymerase—DNA polymerases (which synthesize DNA) and RNA polymerase (which makes RNA). Within these two classes, there are numerous sub-types of polymerase, depending on what type of nucleic acid can function as template and what type of nucleic acid is formed. For example, for whole genome amplification by multiple displacement amplification, highly processive DNA polymerases are used. One example of such a polymerase is Phi29, which can produce DNA fragments of greater than 70 kb, and favors uniform representation of sequences, with very small error rates during amplification.

The term “sample” as used herein relates to a material or mixture of materials, typically, although not necessarily, in fluid form, containing one or more components of interest. Samples include, but are not limited to, biological samples obtained from natural biological sources, such as cells or tissues. The samples also may be derived from tissue biopsies and other clinical procedures.

The term “binding buffer,” as used herein, refers to a buffer solution that can be used to selectively bind nucleic acids to a support, separation medium or filtration apparatus. Binding buffers comprise, for example, aqueous solutions containing Tris, EDTA and salts such as, for example, NaCl, MgCl₂, or CaCl₂.

In this specification and the appended claims, the singular forms “a,”“an,” and “the” include plural reference, unless the context clearly dictates otherwise. Unless defined otherwise, all technical and scientific terms used herein have the same meaning as commonly understood to one of ordinary skill in the art.

The present disclosure describes a method for separating different fragments of nucleic acids present in a sample, on the basis of differences in size. Size fractionation is accomplished by selectively adsorbing nucleic acid fragments onto a glass fibers. Under certain solvent conditions, large nucleic acid fragments are preferentially bound to the glass fibers, while small fragments do not bind and pass through the filtration column in the flow-through fraction. The cut-off size (i.e. the size of bound nucleic acid vs. the size of nucleic acid not bound to the column) is varied by changing the ionic strength and/or hydrophobicity of the solution.

In one embodiment, a method for separating nucleic acid molecules according to size is provided. In an aspect, the sample contains one or more nucleic acids of different sizes, such as a sample of DNA fragments of different size, for example. In another aspect, the nucleic acid fragments are present in a solution of binding buffer, containing a specific concentration of a chaotropic salt such as guanidine hydrochloride, for example. The chaotropic salt is present when the sample is first applied to the glass fiber filter. In an embodiment, the glass fiber filter or column then selectively binds large nucleic acid fragments at specific salt concentrations. The nucleic acid fragments bound to the glass filter are then eluted using various concentrations of alcohol.

The methods described herein are directed to the preparation of large-sized nucleic acid fragments for use as templates for amplification by multiple strand displacement (MDA). In an embodiment, the sample is combined with a solution of binding buffer containing a chaotropic salt, and the sample is then contacted to a glass fiber filtration column. At specific salt concentrations, only fragments larger than 100 kb are retained on the column. These fragments are then eluted off the column, using varying concentrations of alcohol. The large fragments separated by the methods described herein are then used as templates for MDA by a highly processive enzyme, such as Phi29, for example. In certain embodiments, the nucleic acids fractionated by the methods described herein include, without limitation, fragments of genomic DNA, DNA fragments obtained from tissue biopsy samples, etc.

In one embodiment, nucleic acids in a sample are fractionated according to size using glass fiber filtration columns. The ability of glass to bind DNA is known in the art. See, e.g., Vogelstein et al., Proc. Natl. Acad. Sci. 76: 615-619 (1979). In an aspect, the methods use two-layer glass fiber filters. The two-layer filters consist of two filter layers in a single housing. The first layer is a coarse pre-filter that removes larger particulate matter commonly found in biological samples. The second layer is a membrane filter that selectively binds nucleic acid fragments. The filters comprise borosilicate glass fibers housed in an inert material, such as polycarbonate or polypropylene, for example. The borosilicate fibers of the filter are chemically inert and resistant to most solvents. The fibers of the filter have a rigid structure that increases the surface area of the filter and provides better retention for nucleic acid molecules. Glass fiber filters of the type described here are commercially available.

Methods for fractionation of a sample containing nucleic acids of different sizes are described herein. Size fractionation is accomplished by selectively adsorbing nucleic acid fragments from the sample onto a glass fiber filtration column. The size of nucleic acids is generally described by the number of nucleotides, expressed in kilobases (kb), and/or by molecular weight. In one aspect, the nucleic acid fragments separated by the methods described herein have a size of at least 10 kb, preferably ranging from about 10 kb to about 50 kb, and more preferably, greater than 100 kb, but less than 300 Mb. In an embodiment, the methods are used to selectively separate DNA fragments at least 100 kb in size.

In the methods described herein, nucleic acid fragments of a desired size are separated from the sample mixture using a glass fiber filter and varying the ionic strength. In an embodiment, the ionic strength is varied by the addition of chaotropic salts. A chaotropic salt is a compound that has the ability to disrupt the regular hydrogen bond structures in a non-polar molecule present in aqueous solution, thereby increasing the solubility of the non-polar molecule in water. Chaotropic salts include, but are not limited to, guanidine hydrochloride (GHC), potassium iodide (KI), sodium iodide (NaI), etc. Other guanidine salts are also possible chaotropes. The interaction of nucleic acid molecules with water molecules present in aqueous solution can impede binding of the nucleic acid to the glass fiber surface, because the solvent molecules occupy all the accessible binding sites on the nucleic acid molecule. Chaotropic salts, such as GHC, for example, are used to decrease binding of solvent water molecules to nucleic acids, thereby freeing up sites on the nucleic acid molecule for binding to the surface of the glass fibers.

In an embodiment, nucleic acid fragments bound to the glass fiber filtration column are eluted by washing with a solution that increases hydrophobicity. In an aspect, the wash solution is more hydrophobic than the binding buffer containing the chaotropic salt. Wash solutions include, but are not limited to, alcohols, e.g., lower alcohols, such as ethanol or isopropyl alcohol, for example. In certain embodiments, the washing step is followed by elution with a low ionic strength buffer, such as TE buffer, for example. Small nucleic acid fragments, which are not strongly retained on the glass fiber column even at high ionic strength, are eluted from the column first, while larger nucleic acid fragments remain bound to the column. As the hydrophobicity is increased, larger nucleic acid fragments are progressively eluted at higher alcohol concentrations. In certain embodiments, the elution step is omitted and the small fragments that pass into the flow-through fraction are collected, while the column, with larger fragments still bound to it, is discarded.

In an embodiment, different combinations of ionic strength and hydrophobicity are used to alter the binding of DNA fragments to the glass fiber filters, based on size of the DNA fragments. For example, at low salt concentration (i.e. 0M of chaotropic salt) and 0-40% alcohol concentration, fragments in the 10 kb size range are bound to the column, and subsequently eluted. As the salt and alcohol concentrations are varied, different size fractions are bound and then eluted from the columns. For example, increasing amounts of small fragments (<10 kb) can be recovered from the sample mixture at higher salt and/or alcohol concentrations (i.e. 2M of chaotropic salt and 40% alcohol. Similarly, intact high molecular weight (>10 kb) fractions can be recovered from the sample mixture by varying the salt and alcohol concentrations (i.e. 500 mM of chaotropic and 10% alcohol) used to bind and then wash the columns.

In one embodiment, a method for separating nucleic acid molecules according to size, from a sample containing nucleic acid of different size is described herein. In an aspect, the sample is a solution of DNA or RNA fragments obtained from a clinical procedure. In another aspect, the sample is a mixture of DNA fragments of different sizes obtained from a tissue biopsy. A sample of this type can contain DNA or RNA fragments with a wide variety of sizes, ranging from fragments as small as 1 kb to fragments as large as 500 Mb.

In another embodiment, the methods are used to simultaneously collect both high and low molecular weight nucleic acid fragments for use in different downstream applications. At specific ionic strength or hydrophobicity, large DNA or RNA fragments are bound to the glass fiber column, while small DNA or RNA fragments are not bound and can be collected in the flow-through fraction. By varying the ionic strength/hydrophobicity conditions, flow-through fractions containing nucleic acid fragments of different sizes are collected. The smaller DNA fragments are used in applications like PCR-based sequencing, or somatic genotyping using polymorphic markers such as SNPs or VTRs, for example. The larger DNA fragments are used for enzymatic amplification and aCGH analyses.

In various embodiments, the methods described herein are used to purify templates for whole genomic amplification. In an aspect, the templates comprise large molecular weight DNA fragments for use in enzymatic amplification techniques using highly processive enzymes.

Enzymatic amplification uses highly processive DNA polymerases, which synthesize DNA sequences by multiple strand displacement (MDA). This method can generate thousands of high molecular weight copies of genomic DNA without using ligation or thermocycling. Furthermore, MDA favors uniform representation of sequences because each priming event takes place over a very long stretch of the genome. Highly processive enzymes can be used to amplify DNA samples from tissue biopsies, and DNA from highly purified cell populations obtained using methods such as laser capture microdissection (LCM) or flow cytometry.

Highly processive enzymes require high molecular weight DNA fragments as templates for linear amplification and accurate representation of the genome of interest. Therefore, the presence of degraded DNA of various sizes in a sample of interest can inhibit the effectiveness of highly processive enzymes in amplification of genomic DNA for assays like aCGH.

Phi29 DNA polymerase is a 66 kDa monomeric DNA polymerase, derived from the Phi29 bacteriophage. During replication, Phi29 catalyzes the formation of a specific protein (terminal protein, TP) that acts as a primer for replication. As replication progresses, Phi29 dissociates from TP and becomes associated with the one strand of the double-stranded DNA that acts as the template. A single Phi29 DNA polymerase molecule can replicate each DNA strand without dissociation from the template, while simultaneously causing displacement of the non-template strand. Phi29 therefore combines high processivity with strand displacement ability, and can produce DNA fragments greater than 70 kb in size, as described in Blanco et al., J. Biol. Chem. 264: 8935-8940 (1989). Furthermore, Phi29 is a linear DNA polymerase and therefore favors uniform representation of sequences during amplification, in contrast to non-linear polymerases like Taq polymerase. In addition, Phi29 has 3′→5′ exonuclease activity and therefore synthesizes DNA with high fidelity, and produces very low error rates during amplification. These properties form the basis of the application of Phi29 amplification of genomic DNA to a number of downstream techniques requiring amplification of DNA, such as aCGH, for example.

If there are breaks or nicks in the genomic DNA template, however, Phi29 is easily dissociated from the template and cannot produce uniformly represented sequences or amplify fragments with high fidelity. This is a significant problem with clinical tissue biopsy samples, which are typically mixtures of undegraded DNA (i.e. DNA of extended length with very few nicks), along with a wide range of smaller, degraded DNA fragments with thousands of nicks or breaks. Using Phi29 DNA polymerase to amplify such samples is unlikely to produce uniformly represented sequences that can be used for genomic analysis techniques like aCGH, for example.

Accordingly, the methods described herein are directed to “cleaning up” the genomic DNA obtained from a clinical sample by selectively separating out large DNA fragments from the sample and then using the separated high molecular weight fragments as templates for Phi29 amplification. In an embodiment, a clinical sample containing a wide range of nucleic acids of different sizes is obtained. The sample is digested with restriction enzymes and then heat inactivated. The heat-inactivated nucleic acid is then mixed with a solution of binding buffer containing a specific concentration of a chaotropic salt, such as guanidine hydrochloride (GHC). The concentration of GHC ranges from 0M to 4M (for example, GHC concentrations of 25 mM, 50 mM, 125 mM, 250 mM, 500 mM, 1M, or 2M). The nucleic acid solution is then transferred to a size separation medium such as a glass fiber filtration column, for example. Ethanol is added to the column, at concentrations ranging from 0% to 100% (for example, ethanol concentrations of 0% (i.e. water), 10%, 20%, 30%, 40%, or 50%). At a specific concentration of GHC, high molecular weight nucleic acid fragments are preferentially bound to the glass fiber filtration column. These high molecular weight fragments are eluted off the column using a specific concentration of ethanol, and the eluted fragments are used as templates for amplification by Phi29.

The present disclosure includes a method for enriching genomic DNA samples with high molecular weight fragments. Such enriched samples are particularly useful in techniques that require a minimum amount of starting material including techniques such as aCGH, for example. In various embodiments, genomic DNA is prepared for use in CGH employing the size fractionation methods described above. In an aspect, genomic DNA is amplified using highly processive enzymes, for use in CGH analyses.

CGH was initially a molecular cytogenetic method capable of detecting and mapping relative DNA sequence copy numbers between genomes, as disclosed by Kallioniemi et al., Science 258: 818-821 (1992). Briefly, total tumor DNA is biotinylated and normal genomic reference DNA is labeled with dioxigenin. The two are then simultaneously hybridized to normal metaphase spreads, using unlabeled Cot-1 DNA for blocking. Hybridization of tumor DNA is detected with a green fluorophore (FITC-avidin, for example), while reference DNA hybridization is detected using a red fluorophore (i.e. rhodamine antidioxigenin). The relative amounts of tumor and reference DNA that bind to a particular location on the chromosome are dependent on the relative abundance of tumor and reference DNA sequences in the samples. Therefore, the ratio of tumor DNA to normal reference DNA can be determined by measuring the ratio of green-to-red fluorescence. The fluorescence signals can be quantitated using digital image analysis methods. An elevated green-to-red ratio signifies gene amplification or chromosomal duplication in the tumor DNA, whereas a reduced ratio indicates chromosomal loss or deletion. CGH can quantitatively distinguish a change in copy number of plus or minus one chromosome, and therefore is an effective tool for screening DNA samples from solid tumors for physical deletions or mutations.

A significant limitation of conventional CGH is the use of metaphase chromosome spreads, which limits detection to events occurring over very small regions of the chromosome (usually less than 20 Mb). This limitation can be overcome by hybridizing the sample and reference DNA to microarrays, in a technique known as array-based comparative genomic hybridization (aCGH). This technique has a number of advantages such as higher resolution and dynamic range, direct mapping of chromosomal aberrations and higher throughput than conventional CGH. See Pinkel et al., Nature Genetics 20: 207-211 (1998).

Array-based CGH is now widely used to study chromosomal aberrations. A number of different platforms have been used for CGH-type analyses of mammalian genomes. These methods use arrays containing large insert genomic clones, such as bacterial artificial chromosomes (i.e. BAC arrays), cDNA arrays and oligonucleotide arrays. These techniques require a minimum amount of starting material, i.e. about 20 μg of genomic DNA, to ensure complete coverage and representation of the genome in question.

An aCGH experiment usually requires about 10 μg of genomic DNA as starting material, for complete coverage and representation of the target genome. One way to extend the limited amount of genomic DNA available from a clinical sample is by enzymatic amplification, or multiple strand displacement amplification (MDA) of the genomic DNA. Highly processive DNA polymerases (as discussed below) are typically used for such amplifications, and these enzymes require high molecular weight DNA fragments (i.e. greater than 10 kb) as a template for linear amplification. Accordingly, in an embodiment, a method for separating DNA fragments at least 10 kb in length from a sample containing a mixture of DNA fragments of different sizes is provided. In another embodiment, the DNA fragments separated by the method described herein range in size from about 50 kb to about 100 kb. In an embodiment, the methods are used to isolate DNA fragments with sizes of greater than 100 kb, but less than 300 Mb.

The methods described herein are used in conjunction with many downstream applications. The current methods are used to separate and collect both high and lower molecular weight fragments from a single sample, for use in different applications. The low molecular weight fractions are used in a variety of assays such as, for example, PCR-based sequencing and somatic genotyping with polymorphic markers such as single nucleotide polymorphism (SNP), or VTRs. Small nucleic fragments are also useful in other applications, such as sequence-specific modification or editing of genomic DNA (using small-fragment homologous replacement (SFHR), for example).

In embodiments, the present disclosure includes kits for the separation of nucleic acids on the basis of size. The kits contain at least one suitably packaged glass fiber filtration column, along with solutions of binding buffer. In embodiments, the kits described herein contain stock solutions of reagents necessary for size separation, i.e. chaotropic salts and alcohols. The stock solutions of the kit can be used to make solutions of salt and alcohol of various concentrations. The kit may also contain instructions providing information on the use of the glass fiber filtration column to separate nucleic acids according to size. In embodiment, the kits may further contain highly processive enzymes for use in preparing large-sized nucleic acid templates for amplification and use in downstream applications.

### EXAMPLES

### Example 1

### Size Fractionation of DNA by Glass Fiber Filters

For purposes of this example, large molecular weight DNA fragments for use as amplification templates were prepared as follows:

To create a DNA sample consisting of a wide range of DNA sizes, 1 mg samples of calf thymus DNA were either digested with AluI (4-base recognition sequence) or EcoRI (6-base recognition sequence) in a 2 mL reaction. A third sample consisting of undigested DNA was resuspended in 2 mL of water. The samples were then incubated at 37° C. for 2 hours, and then heat inactivated at 65° C. for 20 minutes. The digested and undigested DNA samples were then mixed in the ratio 4:1:1 AluI digest: EcoRI digest: undigested. The final DNA concentration was 500 ng/mL.

A 150 μL aliquot, containing 15 μg of the heat inactivated DNA mixture, was added to an equal volume of a “binding buffer” with increasing concentrations of GHC (0M to 4M) in the presence of 50 mM bis-Tris pH 6.6 and 10 mM EDTA. The final volume was 300 μL. The mixtures (0-2M final GHC concentration) were then transferred to two-layer glass fiber spin columns (with 6 columns for each GHC concentration). An equal volume of 0% ethanol (water), 20%, 40%, 60%, 80% and 100% ethanol were then added to each set of six columns (final concentrations of 0-50% ethanol). The samples were briefly vortexed and centrifuged for 1 minute at maximum speed (>13,000 rpm). Each filter was washed with 80% ethanol (500 μL). The DNA bound to the glass fiber spin column was eluted using 50 μL 0.1% Sarcosyl, 10 mM Tris pH 8.0 and 1 mM EDTA. 1 μL of each eluate was then run on a DNA 12000 Lab Chip (Agilent Technologies).

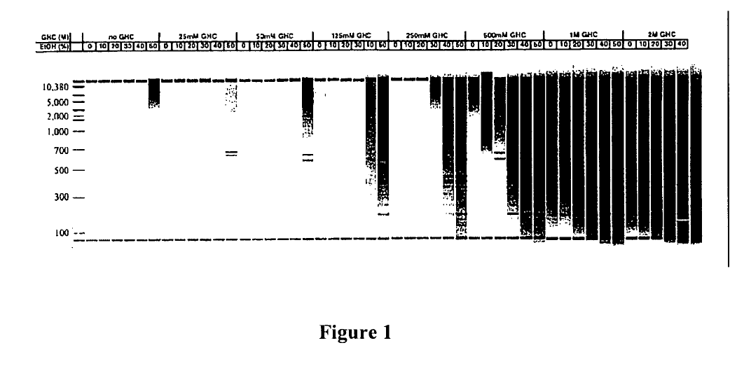

FIG. 1 shows the results of a size fractionation experiment. At low salt (0M GHC) and 0-40% alcohol concentrations, fragments in the 10 kb size range are bound to and subsequently eluted from the column. As the salt and alcohol concentrations are varied, different size fractions are bound and then eluted from the columns. For example, increasing amounts of small fragments (<10 kb) can be recovered from the DNA mixture at higher salt and/or alcohol concentrations (i.e. 2M GHC and 40% ethanol). Similarly, intact high molecular weight (>10 kb) fractions can be recovered from the DNA mixture by varying the salt and alcohol concentrations (i.e. 500 mM GHC and 10% ethanol) used to bind and then wash the columns.

The various embodiments described above are provided by way of illustration only and should not be construed to limit the claims. Those skilled in the art will readily recognize various modifications and changes that may be made to the present methods without following the example embodiments and applications illustrated and described herein, and without departing from the true spirit and scope of the following claims.

1. A method for fractionating nucleic acid molecules according to size, comprising:
preparing a buffered nucleic acid composition by combining a sample of nucleic acids with a solution of binding buffer;
contacting the buffered nucleic acid composition to a glass fiber filtration medium; and
washing the glass fiber filtration medium with a wash medium to fractionate the nucleic acid sample according to size,
the binding buffer including a chaotropic salt; the wash medium comprising a lower alcohol, and the concentration of the chaotropic salt and lower alcohol being selected to separate a nucleic acid of a desired size.

2. The method of claim 1, further comprising recovering a large-sized nucleic acid fragment, the large-sized fragment serving as a template for amplification by a highly processive enzyme.

3. The method of claim 1, wherein the nucleic acid sample is isolated from a tissue biopsy.

4. The method of claim 1, wherein the chaotropic salt is guanidine hydrochloride.

5. The method of claim 1, wherein the concentration of the chaotropic salt ranges from 0M to 4M, in increments of 25 mM.

6. The method of claim 1, wherein the concentration of ethanol ranges from 0% to 100%, in increments of 10%.

7. The method of claim 1, wherein the eluted nucleic acid has a size of at least 10 kb.

8. The method of claim 1, wherein the eluted nucleic acid has a size from about 50 kb to about 100 kb.

9. The method of claim 1, wherein the eluted nucleic acid has a size of greater than 100 kb but less than 300 Mb.

10. The method of claim 2, wherein the highly processive enzyme is Phi29.

11. A method for enriching a sample of genomic DNA with DNA fragments of a desired size, comprising:
preparing a DNA sample containing one or more DNA fragments of different sizes in a solution of binding buffer containing a chaotropic salt;
contacting the DNA sample to a glass fiber filtration medium;
washing the glass fiber filtration medium with alcohol; and
eluting the DNA fragments bound to the separation medium,
the concentrations of chaotropic salt and alcohol being selected to separate DNA fragments of a desired size.

12. The method of claim 11, wherein eluted DNA fragments with size greater than 100 kb but less than 300 Mb are used in array-based comparative genomic hybridization.

13. The method of claim 11, wherein the DNA fragments with size less than 10 kb remaining in the flow-through fraction are used in PCR-based downstream applications.

14. The method of claim 11, wherein DNA fragments with size greater than 10 kb comprise a template for amplification by a highly processive enzyme.

15. The method of claim 14, wherein the highly processive enzyme is Phi29.

16. A kit for size separation of a nucleic acid sample, comprising:
at least one glass fiber filtration column;
a binding buffer used to apply a nucleic acid sample to the glass fiber filtration column; and
a wash solution for removing the nucleic acid sample from the glass fiber filtration column.

17. The kit of claim 16, further comprising a solution of a chaotropic salt.

18. The kit of claim 16, further comprising a highly processive enzyme.
